(19) United States
(12) Patent Application Publication
(10) Pub. No.: US 2024/0325496 A1
(43) Pub. Date: Oct. 3, 2024

(54) CXC CHEMOKINE AGONISTS AND ANTAGONISTS IN COVID-19 DISEASE AND DIAGNOSTIC ASSAYS

(71) Applicants: University of Pittsburgh - Of the Commonwealth System of Higher Education, Pittsburgh, PA (US); The Trustees of Indiana University, Bloomington, IN (US)

(72) Inventors: Cecelia C. Yates, Pittsburgh, PA (US); Monte S. Willis, Indianapolis, IN (US)

(73) Assignees: University of Pittsburgh - Of the Commonwealth System of Higher Education, Pittsburgh, PA (US); The Trustees of Indiana University, Bloomington, IN (US)

(21) Appl. No.: 18/575,429

(22) PCT Filed: Jun. 28, 2022
(86) PCT No.: PCT/US2022/035333

§ 371 Date: Dec. 29, 2023

Related U.S. Application Data
(60) Provisional application No. 63/216,392, filed on Jun. 29, 2021.

(51) Int. Cl. A61K 38/21; A61K 31/573; A61K 31/706; A61K 33/00; A61K 38/10; A61K 47/36; C07K 16/10; G01N 33/50; G01N 33/68

(52) U.S. Cl. CPC A61K 38/217; A61K 31/573; A61K 31/706; A61K 33/00; A61K 38/10; A61K 47/36; C07K 16/1003; G01N 33/5091; G01N 33/6893; G01N 2333/165

(57) ABSTRACT

Methods of treating a disease, disorder or syndrome associated with a severe acute respiratory syndrome coronavirus 2 (SARS-CoV-2) infection (such as COVID-19) in a subject by administering one or more C-X-C chemokine receptor 3 (CXCR3) antagonist peptides are described. Diagnostic methods for enhancing replication of SARS-CoV-2 in cultured cells and/or detecting the presence of SARS-CoV-2 in cultured cells by contacting the cells with a CXCR3 agonist peptide are also described.

### CROSS REFERENCE TO RELATED APPLICATIONS

This application claims the benefit of U.S. Provisional Application No. 63/216,392, filed Jun. 29, 2021, which is herein incorporated by reference in its entirety.

### FIELD

This disclosure concerns C-X-C chemokine receptor 3 (CXCR3) antagonist and agonist peptides and their use for the treatment of conditions associated with coronavirus disease 19 (COVID-19) and in diagnostic assays for the detection of severe acute respiratory syndrome coronavirus 2 (SARS-CoV-2).

### BACKGROUND

Coronavirus disease 2019 (COVID-19) is a viral pneumonia caused by severe acute respiratory syndrome coronavirus 2 (SARS-CoV-2), the virus responsible for a worldwide pandemic with over 500 million cases, including over 6.3 million fatalities. COVID-19 patients have a broad array of clinical manifestations, from asymptomatic to severe symptoms, such as acute respiratory distress syndrome (ARDS), pro-thrombotic states, multi-organ failure, a sepsis-like syndrome (and associated cytokine storm), and fibrosis related death. Viral damage and the multi-organ hyperinflammatory state, often referred to as the “cytokine storm,” is thought to cause tissue damage through repetitive insult and induce a continuously remodeling fibrotic state. However, modeling and understanding underlying triggers of this dynamic and complex disease state have proven difficult. One study indicated that patients with severe-to-critical COVID-19 have a dysregulated bronchoalveolar immune landscape caused by a cytokine storm, which includes high levels of IL-6, IL-1, and CXCL10. The CXC chemokine CXCL10 has also been implicated in the initiation and propagation of the cytokine storm in COVID-19. Moreover, CXCL10 has been identified as modulating pathways involved in COVID-19 severity and pulmonary fibrosis. Patients with COVID-19 also have elevated circulating CXCL10 and a correlation exists between CXCL10 levels and disease severity and viral loads. There is an unmet urgent need for therapies that mitigate severe COVID-19 and the underlying organ fibrotic insult.

### SUMMARY

Provided herein are methods of treating a disease, disorder or syndrome associated with a severe acute respiratory syndrome coronavirus 2 (SARS-CoV-2) infection in a subject. In some implementations, the method includes administering to the subject a therapeutically effective amount of a composition that includes at least one C-X-C chemokine receptor 3 (CXCR3) antagonist peptide.

Also provided herein are in vitro methods for enhancing replication of SARS-CoV-2 in a cell. In some implementations, the method includes contacting the cell with an effective amount of a CXCR3 agonist peptide.

Further provided herein are methods for detecting the presence of SARS-CoV-2 in a biological sample. In some implementations, the method includes contacting cultured cells susceptible to infection by SARS-CoV-2 with the biological sample under conditions sufficient to allow for infection of the cultured cells if SARS-CoV-2 is present in the biological sample; contacting the cultured cells with a CXCR3 agonist peptide; and detecting SARS-CoV-2 in the cultured cells.

The foregoing and other objects and features of the disclosure will become more apparent from the following detailed description, which proceeds with reference to the accompanying figures.

### BRIEF DESCRIPTION OF THE DRAWINGS

FIGS. 1A-1P: Expression of IP-10 in human lung, heart, and skeletal muscle by immunohistochemical analysis. (FIGS. 1A-1F) IP-10 expression in representative sections of lung (at 10×) from tissue shown in FIGS. 1G-1H (IX). (FIGS. 1I-IL) IP-10 expression from representative sections of heart (10×). (FIGS. 1M-1N) IP-10 expression in striated muscle from trachea at 10× and 1×, respectively. (FIG. 10) IP-10 IHC positive control on human prostate (primary antibody: anti-IP-10 (Rb IgG1) and secondary antibody: anti-Rb HRP). (FIG. 1P) IP-10 negative control on human prostate (primary antibody: Rb IgG1 polyclonal and secondary antibody: anti-Rb HRP). FIGS. 1A, 1C, 1E, 1G, 1I, 1K were taken from a single non-COVID-19 patient. FIGS. 1B, 1D, 1F, 1H, 1J, 1L, 1M, 1N were taken from a single PCR-confirmed COVID-19 patient.

### SEQUENCE LISTING

The amino acid sequences listed in the accompanying sequence listing are shown using standard three letter code for amino acids, as defined in 37 C.F.R. 1.822. The Sequence Listing is submitted as an ASCII text file, created on Jun. 22, 2022, 10.9 KB, which is incorporated by reference herein. In the accompanying sequence listing:

SEQ ID NOs: 1-4 are the amino acid sequences of IP-10 peptides and variants.

SEQ ID NOs: 5-42 are the amino acid sequences of in silico designed peptides.

### DETAILED DESCRIPTION

### I. Abbreviations

CoV coronavirus
COVID-19 coronavirus disease 2019
CXCL10 C-X-C chemokine ligand 10
CXCL11 C-X-C chemokine ligand 11
CXCR3 C-X-C chemokine receptor 3
HF heart failure
IHC immunohistochemistry
IP-10 interferon-inducible protein 10
MTD maximum tolerated dose
SARS severe acute respiratory syndrome

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various implementations of the disclosure, the following explanations of specific terms are provided:

Aerosol: A suspension of fine solid particles or liquid droplets in a gas (such as air).

Administration: The introduction of a composition (such as a protein or peptide) into a subject by a chosen route. For example, if the chosen route is intravenous, the composition is administered by introducing the composition into a vein of the subject. Exemplary routes of administration include, but are not limited to, injection (such as intraocular, subcutaneous, intramuscular, intradermal, intraperitoneal, and intravenous), oral, intraductal, sublingual, transdermal, intranasal, topical, inhalation routes and via a medical implant.

Agonist: A drug or molecule (such as a peptide) that promotes the activity or function of another drug or molecule. For example, an agonist of a receptor is a molecule that enhances activity (such as signaling activity) of the receptor. In some implementations of the present disclosure, the “CXCR3 agonist” is a peptide that binds CXCR3 and enhance its signaling activity.

Antagonist: A drug or molecule (such as a peptide) that interferes with or inhibits the action or function or another drug or molecule. For example, an antagonist of a receptor is a molecule that inhibits activity (such as signaling activity) of the receptor. As used herein, an “antagonist of CXCR3 signaling” refers to a peptide that interferes with the signaling activity mediated by CXCL10 and/or CXCR3. In some examples, the antagonistic peptides disclosed herein bind CXCL10 or CXCL11 and prevent binding of these proteins to their receptor CXCR3. In other examples, the antagonistic peptides bind CXCR3 and prevent binding of one or more ligands for CXCR3.

Conservative variants: “Conservative” amino acid substitutions are those substitutions that do not substantially affect or decrease an activity or antigenicity of a protein, such as an IP-10 peptide, a CXCR3 antagonist peptide or a CXCR3 agonist peptide. For example, the peptides of any one of SEQ ID NOs: 1-42 can include at most about 1, at most about 2, at most about 3, at most about 4, at most about 5, at most about 6, at most about 7 or at most about 8 conservative substitutions (such as 1, 2, 3, 4, 5, 6, 7 or 8) conservative substitutions, such as 1 to 3, 1 to 5 or 2 to 6 conservative substitutions, and retain biological activity, such as the ability to bind CXCR3, CXCR4, CXCL4, CXCL9, CXCL10 and/or CXCL11, and/or the ability to activate CXCR3. In particular examples, the peptide variants have no more than 3 conservative amino acid substitutions. Specific, non-limiting examples of a conservative substitution include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variant also includes the use of a substituted amino acid in place of an unsubstituted parent amino acid. Non-conservative substitutions are those that reduce an activity or antigenicity.

Contacting: Placement in direct physical association; includes both in solid and liquid form.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2, and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), human coronavirus NL63 (NL63-CoV).

COVID-19: The disease caused by the coronavirus SARS-CoV-2.

CXCL10 (C-X-C chemokine ligand 10): A chemokine of the CXC subfamily and ligand for the receptor CXCR3. Binding of CXCL10 to CXCR3 results in pleiotropic effects, including stimulation of monocytes, natural killer and T-cell migration, modulation of adhesion molecule expression, and inhibition of vessel formation. CXCL10 is also known as interferon-γ-inducible 10 kDa protein (IP-10).

CXCR3 (C-X-C chemokine receptor 3): A G protein-coupled receptor with selectivity for four chemokines, CXCL4, CXCL9, CXCL10 and CXCL11. Binding of chemokines to CXCR3 induces signaling and cellular responses that are involved in leukocyte trafficking, most notably integrin activation, cytoskeletal changes and chemotactic migration.

Cytokine storm syndrome: A severe immune reaction in which the innate immune system causes uncontrolled and excessive release of cytokines into the blood. Excessive production of proinflammatory cytokines can aggravate existing respiratory distress, as well as cause overwhelming systemic inflammation, hemodynamic instability, multiple organ dysfunction, and potentially death. Cytokine storm syndrome is also called hypercytokinemia. Detection of cytokine storm syndrome in a patient can be accomplished using standard diagnostic methods, including but not limited to elevation of plasma C-reactive protein (CRP) levels, elevation of interleukin-6 (IL-6) levels, abnormalities of markers of blood clotting such as D-dimer or fibrinogen and elevated ferritin levels. Protocols for detecting cytokine storm in a COVID-19 patient are known and described, for example, in Soy et al., Clin Rheumatol., 39(7):2085-2094, 2020. Additional information concerning cytokine storm syndrome can be found, for example, in Ye et al, Journal of Infection, 80(6):607-613, 2020.

Fibrosis: A condition associated with the thickening and scarring of connective tissue. Often, fibrosis occurs in response to an injury, such as from a disease or condition that damages tissue. Fibrosis is an exaggerated wound healing response that when severe, can interfere with normal organ function. Fibrosis can occur in almost any tissue of the body, including in the lung (pulmonary fibrosis, cystic fibrosis, radiation-induced lung injury), liver (cirrhosis, biliary atresia), heart (arterial fibrosis, endomyocardial fibrosis, prior myocardial infarction), brain, skin (scleroderma, sclerosis), kidney, joints and intestine (Crohn's disease).

Heart failure: A disease resulting from the inability of the heart to pump blood in sufficient quantities to meet the body's requirements. Common causes of heart failure include coronary heart disease, previous myocardial infarction, high blood pressure, atrial fibrillation, valvular heart disease, excess alcohol use, infection and cardiomyopathy. There are two main types of heart failure—heart failure due to left ventricular dysfunction and heart failure with normal ejection fraction—depending on whether the ability of the left ventricle to contract is affected or the heart's ability to relax is affected.

Ischemia: A vascular phenomenon in which a decrease in the blood supply to a bodily organ, tissue, or part is caused, for instance, by constriction or obstruction of one or more blood vessels. Ischemia sometimes results from vasoconstriction or thrombosis or embolism. Ischemia can lead to direct ischemic injury, tissue damage due to cell death caused by reduced oxygen supply.

Ischemia-reperfusion injury: Tissue damage caused by the return of blood supply after a period of ischemia or lack of oxygen. The restoration of blood flow results in inflammation and oxidative damage through the induction of oxidative stress.

Isolated: An “isolated” or “purified” biological component (such as a nucleic acid or peptide) has been substantially separated, produced apart from, or purified away from other biological components in the cell of the organism in which the component occurs, that is, other chromosomal and extra-chromosomal DNA and RNA, and proteins. Nucleic acids, peptides and proteins that have been “isolated” or “purified” thus include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term.

Peptide or polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide,” “peptide,” or “protein” as used herein are intended to encompass any amino acid sequence and include modified sequences such as glycoproteins. The terms “polypeptide” and “peptide” are specifically intended to cover naturally occurring proteins, as well as those which are recombinantly or synthetically produced.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the peptides herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. For topical application to the eye, agents can be mixed, for example, with artificial tears and other emulsions.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in pro-inflammatory cytokines. “Ameliorating” refers to the reduction in the number or severity of signs or symptoms of a disease, such as a coronavirus infection.

SARS-CoV-2: A coronavirus of the genus betacoronavirus that first emerged in humans in 2019. This virus is also known as Wuhan coronavirus, 2019-nCoV, or 2019 novel coronavirus. The term “SARS-CoV-2” includes variants thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2) and omicron (B.1.1.529 and BA lineages). Symptoms of SARS-CoV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-CoV-2 virion includes a viral envelope with large spike glycoproteins. The SARS-CoV-2 genome, like most coronaviruses, has a common genome organization with the replicase gene included in the 5-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a particular polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. In addition, Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 90%, 95%, 96%, 97%, 98% or 99% sequence identity counted over the full length alignment with the amino acid sequence of the polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals. In some examples, the subject has a coronavirus infection, such as a SARS-CoV-2 infection.

Synthetic: Produced by artificial means in a laboratory, for example a synthetic nucleic acid or peptide can be chemically synthesized in a laboratory.

Therapeutically effective amount: A quantity of a specified agent (such as a CXCR3 antagonist peptide or a CXCR3 agonist peptide) sufficient to achieve a desired effect in a subject, cell or culture being treated with that agent. In some implementations, the therapeutically effective amount is the amount of peptide necessary to inhibit CXCR3 signaling. In other implementations, the therapeutically effective amount is the amount of peptide sufficient to treat or ameliorate a disease, disorder or syndrome associated with SARS-CoV-2 infection and/or COVID-19 in a subject.

### III. Methods of Treating COVID-19 and Associated Syndromes

COVID-19, caused by the SARS-CoV-2 betacoronavirus, has affected millions worldwide and one fifth of infected people have developed fibrotic tissue in the lung. Patients with severe-to-critical COVID-19 infection have a dysregulated bronchoalveolar immune landscape caused by a cytokine storm, which includes high levels of IL-6, IL-1, and CXCL10. As a result of the severe inflammatory response in COVID-19 disease, counter inflammatory pathways are enacted to heal and repair the tissue, prolonging the fibrotic response and the formation of pulmonary fibrosis. To prevent lung damage in fibrosis, it is important to understand the underlying pathways that parallel, but are not necessarily equal to, virus clearance.

It is hypothesized that novel anti-fibrotic therapies designed for idiopathic pulmonary fibrosis (IPF) could not only block TGF-β pathways, but also act to inhibit viral infection. CXCL10 modulates pathways involved in COVID-19 severity and pulmonary fibrosis, suggesting this chemokine is a promising target for treating viral-mediated pro-fibrotic pathways. A series of biometric peptides that modulate CXCL10 have been designed (see Example 2). These peptides dually target multiple mechanisms of CXCL10 in hyperinflammatory and fibrotic disease states. It has been shown that CXCL10 mimetic peptides inhibit the activity of pro-fibrotic TGF-β. The present disclosure proposes to treat the convergent disease mechanisms in pulmonary fibrosis and COVID-19 using the CXL10 antagonist peptides disclosed herein and described in US 2019/0298802, which is herein incorporated by reference in its entirety.

Provided herein is a method of treating a disease, disorder or syndrome associated with a SARS-CoV-2 infection in a subject. In some implementations, the method includes administering to the subject a therapeutically effective amount of a composition that includes at least one C-X-C chemokine receptor 3 (CXCR3) antagonist peptide. In some examples, the CXCR3 antagonist peptide is 12 to 30 amino acids in length and includes at least 12, at least 13, at least 14, at least 15, at least 16, at least 17, at least 18, at least 19 or at least 20 consecutive amino acids of any one of SEQ ID NOs: 1-6, 10, 11, 20 and 24-41. In some examples, the CXCR3 antagonist peptide is 14 to 25 amino acids in length and includes at least 14, at least 15, at least 16, at least 17, at least 18, at least 19 or at least 20 consecutive amino acids of any one of SEQ ID NOs: 1-6, 10, 11, 20 and 24-41. In some examples, the CXCR3 antagonist peptide is 16 to 20 amino acids in length and includes at least 16, at least 17, at least 18, at least 19 or at least 20 consecutive amino acids of any one of SEQ ID NOs: 1-6, 10, 11, 20 and 24-41.

In particular examples, the amino acid sequence of the CXCR3 antagonist peptide is at least 90%, at least 95%, at least 96%, at least 97%, at least 98% or at least 99% identical to any one of SEQ ID NOs: 1-6, 10, 11, 20 and 24-41. In specific non-limiting examples, the amino acid sequence of the CXCR3 antagonist peptide comprises or consists of any one of SEQ ID NOs: 1-6, 10, 11, 20 and 24-41.

In some implementations, the method further includes selecting a subject with a current or prior SARS-CoV-2 infection for treatment. In some examples in which the subject had a prior SARS-CoV-2 infection, the infection occurred within the last six months, within the last five months, within the last four months, within the last three months, within the last two months or within the last month.

In some implementations, the subject has been diagnosed with coronavirus disease 19 (COVID-19).

In some implementations, the subject has increased expression of IP-10 relative to a healthy control subject or relative to prior to infection with SARS-CoV-2. For example, the subject may have increased expression of IP-10 in one or more organs or tissues, such as in one or more of the heart, lung, muscle, kidney or liver.

In some implementations, the disease, disorder or syndrome associated with infection by SARS-CoV-2 includes sepsis, cytokine storm syndrome, cardiomyopathy, heart failure, respiratory failure, liver failure, kidney failure, multi-organ failure, fibrosis, pathological tissue remodeling, gastrointestinal inflammation, hypercoagulation, thrombocytopenia, thrombosis, ischemia, ischemia/reperfusion injury, conjunctivitis, keratitis, delirium, neuropathy, or any combination thereof. In some examples, fibrosis is fibrosis of the heart, lung, kidney or skin. In some examples, the disease, disorder or syndrome associated with infection by SARS-CoV-2 includes heart failure.

In some implementations, the composition administered to the subject includes at least two, at least three, at least four, at least five, at least six, at least seven or at least 8 different CXCR3 antagonist peptides. For example, the composition can include 1 to 8 peptides, 2 to 7 peptides, 3 to 6 peptides, or 4 to 5 peptides.

In some implementations, the composition further includes a pharmaceutically acceptable carrier and/or a carrier protein. In some examples, the carrier protein includes heparin, albumin, gelatin, spray-dried lipid-based microparticles (e.g., dipalmitylphosphatidylcholine (DPPC) or distearylphosphatidylcholine (DSPC)), polylactic-co-glycolic acid (PLGA), sodium hyaluronate, or oligosaccharide derivative dipalmitylphospphatidylglycerol (DPPG) (for a review of carrier proteins designed for peptide delivery to the lungs, see Cryan, The AAPS Journal 7(1): Article 4, 2005).

In some implementations, the composition is administered as an aerosol. In some examples, the aerosol droplets are between about 1 μm and about 5 μm in diameter, for example about 2 to about 4 m in diameter. In the context of the present disclosure “about 1 μm” includes 0.95 to 1.05 μm and “about 5 μm” includes 4.95 to 5.05 μm. In other examples, the aerosol droplets are less than or equal to 3 μm in diameter, such as about 3 μm, about 2.5 μm, about 2 μm, about 1.5 μm, or about 1 μm in diameter. In other examples, the aerosol droplets are less than or equal to 8 μm in diameter, such as about 8 μm, about 7.5 μm, about 7 μm, about 6.5 μm, about 6 μm, about 5.5 μm, about 5 μm, about 4.5 μm, about 4 μm, or about 3.5 μm.

In some implementations, the composition is administered using a nebulizer. Any nebulizer capable of converting the composition into an aerosol with an appropriate droplet size for delivery to the lung can be used. In some examples, the nebulizer is an AEROECLIPSE® 11 Breath Actuated Nebulizer (BAN), an AirLife Sidestream nebulizer or an AEROGEN® Ultra vibrating mesh nebulizer. In other implementations, the composition is administered using a dry powder inhaler or a metered dose inhaler.

In other implementations, the composition is administered intravenously.

In some implementations, the composition includes about 50 ng/ml to about 1000 ng/ml of peptide, such as about 100 ng/ml to about 500 ng/ml, about 150 ng/ml to about 450 ng/ml, about 200 ng/ml to about 400 ng/ml, or about 250 ng/ml to about 350 ng/ml of peptide.

In some implementations, the peptide includes at least one chemical modification. In some examples, the peptide includes polyethylene glycol (PEG), one or more D-amino acids (d-AA), N-acetylation, lipidization, or B12 conjugation. In other examples, the peptide is cyclized.

In some implementations, the method further includes administering to the subject a monoclonal antibody specific for SARS-CoV-2. In some examples, the monoclonal antibody includes bamlanivimab, casirivimab and/or imdevimab.

In some implementations, the method further includes administering to the subject remdesivir, dexamethasone, supplemental oxygen and/or any other treatment suitable for a patient with COVID-19.

### IV. Use of CXCR3 Agonists for In Vitro and Diagnostic Methods

Also provided herein are in vitro methods for enhancing replication of SARS-CoV-2 in a cell. In some implementations, the method includes contacting the cell with an effective amount of a C-X-C chemokine receptor 3 (CXCR3) agonist peptide. In some examples, the CXCR3 agonist peptide is 12 to 30 amino acids in length and includes at least 12, at least 13, at least 14, at least 15, at least 16, at least 17, at least 18, at least 19 or at least 20 consecutive amino acids of any one of SEQ ID NOs: 7-9, 12-19, 21-23 and 42. In some examples, the CXCR3 agonist peptide is 14 to 25 amino acids in length and includes at least 14, at least 15, at least 16, at least 17, at least 18, at least 19 or at least 20 consecutive amino acids of any one of SEQ ID NOs: 7-9, 12-19, 21-23 and 42. In some examples, the CXCR3 antagonist peptide is 16 to 20 amino acids in length and includes at least 16, at least 17, at least 18, at least 19 or at least 20 consecutive amino acids of any one of SEQ ID NOs: 7-9, 12-19, 21-23 and 42.

In particular examples, the amino acid sequence of the CXCR3 agonist peptide is at least 90%, at least 95%, at least 96%, at least 97%, at least 98% or at least 99% identical to any one of SEQ ID NOs: 7-9, 12-19, 21-23 and 42. In specific non-limiting examples, the amino acid sequence of the CXCR3 agonist peptide comprises or consists of any one of SEQ ID NOs: 7-9, 12-19, 21-23 and 42.

Further provided herein are methods for detecting the presence of SARS-CoV-2 in a biological sample. In some implementations, the method includes contacting cultured cells susceptible to infection by SARS-CoV-2 with the biological sample under conditions sufficient to allow for infection of the cultured cells if SARS-CoV-2 is present in the biological sample; contacting the cultured cells with a CXCR3 agonist peptide; and detecting SARS-CoV-2 in the cultured cells.

In some examples, the CXCR3 agonist peptide is 12 to 30 amino acids in length and includes at least 12, at least 13, at least 14, at least 15, at least 16, at least 17, at least 18, at least 19 or at least 20 consecutive amino acids of any one of SEQ ID NOs: 7-9, 12-19, 21-23 and 42. In some examples, the CXCR3 agonist peptide is 14 to 25 amino acids in length and includes at least 14, at least 15, at least 16, at least 17, at least 18, at least 19 or at least 20 consecutive amino acids of any one of SEQ ID NOs: 7-9, 12-19, 21-23 and 42. In some examples, the CXCR3 antagonist peptide is 16 to 20 amino acids in length and includes at least 16, at least 17, at least 18, at least 19 or at least 20 consecutive amino acids of any one of SEQ ID NOs: 7-9, 12-19, 21-23 and 42.

In particular examples, the amino acid sequence of the CXCR3 agonist peptide is at least 90%, at least 95%, at least 96%, at least 97%, at least 98% or at least 99% identical to any one of SEQ ID NOs: 7-9, 12-19, 21-23 and 42. In specific non-limiting examples, the amino acid sequence of the CXCR3 agonist peptide comprises or consists of any one of SEQ ID NOs: 7-9, 12-19, 21-23 and 42.

### V. Exemplary Clauses

Clause 1. A method of treating a disease, disorder or syndrome associated with a severe acute respiratory syndrome coronavirus 2 (SARS-CoV-2) infection in a subject, comprising administering to the subject a therapeutically effective amount of a composition comprising at least one C-X-C chemokine receptor 3 (CXCR3) antagonist peptide, wherein the peptide is 12 to 30 amino acids in length and comprises at least 12 consecutive amino acids of any one of SEQ ID NOs: 1-6, 10, 11, 20 and 24-41.

Clause 2. The method of clause 1, wherein the amino acid sequence of the CXCR3 antagonist peptide is at least 95% identical to any one of SEQ ID NOs: 1-6, 10, 11, 20 and 24-41.

Clause 3. The method of clause 1 or clause 2, wherein the amino acid sequence of the CXCR3 antagonist peptide comprises or consists of any one of SEQ ID NOs: 1-6, 10, 11, 20 and 24-41.

Clause 4. The method of any one of clauses 1-3, further comprising selecting a subject with a current or prior SARS-CoV-2 infection for treatment.

Clause 5. The method of clause 4, wherein the prior SARS-CoV-2 infection occurred within the last six months.

Clause 6. The method of any one of clauses 1-5, wherein the subject has been diagnosed with coronavirus disease 19 (COVID-19).

Clause 7. The method of any one of clauses 1-6, wherein the subject has increased expression of IP-10.

Clause 8. The method of any one of clauses 1-7, wherein the disease, disorder or syndrome associated with infection by SARS-CoV-2 comprises sepsis, cytokine storm syndrome, cardiomyopathy, heart failure, respiratory failure, liver failure, kidney failure, multi-organ failure, fibrosis, pathological tissue remodeling, gastrointestinal inflammation, hypercoagulation, thrombocytopenia, thrombosis, ischemia, ischemia/reperfusion injury, conjunctivitis, keratitis, delirium, neuropathy, or any combination thereof.

Clause 9. The method of clause 8, wherein fibrosis is fibrosis of the heart, lung, kidney or skin.

Clause 10. The method of any one of clauses 1-8, wherein the disease, disorder or syndrome associated with infection by SARS-CoV-2 comprises heart failure.

Clause 11. The method of any one of clauses 1-10, wherein the composition comprises at least two, at least three, at least four or at least five CXCR3 antagonist peptides.

Clause 12. The method of any one of clauses 1-11, wherein the composition further comprises a carrier protein.

Clause 13. The method of clause 12, wherein the carrier protein comprises heparin.

Clause 14. The method of any one of clauses 1-13, wherein the composition further comprises a pharmaceutically acceptable carrier.

Clause 15. The method of any one of clauses 1-14, further comprising administering to the subject a monoclonal antibody specific for SARS-CoV-2.

Clause 16. The method of clause 15, wherein the monoclonal antibody comprises bamlanivimab, casirivimab and/or imdevimab.

Clause 17. The method of any one of clauses 1-16, further comprising administering to the subject remdesivir, dexamethasone, and/or supplemental oxygen.

Clause 18. An in vitro method for enhancing replication of severe acute respiratory syndrome coronavirus 2 (SARS-CoV-2) in a cell, comprising contacting the cell with an effective amount of a C-X-C chemokine receptor 3 (CXCR3) agonist peptide, wherein the peptide is 12 to 30 amino acids in length and comprises at least 12 consecutive amino acids of any one of SEQ ID NOs: 7-9, 12-19, 21-23 and 42.

Clause 19. A method for detecting the presence of severe acute respiratory syndrome coronavirus 2 (SARS-CoV-2) in a biological sample, comprising:
contacting cultured cells susceptible to infection by SARS-CoV-2 with the biological sample under conditions sufficient to allow for infection of the cultured cells if SARS-CoV-2 is present in the biological sample;
contacting the cultured cells with a C-X-C chemokine receptor 3 (CXCR3) agonist peptide, wherein the peptide is 12 to 30 amino acids in length and comprises at least 12 consecutive amino acids of any one of SEQ ID NOs: 7-9, 12-19, 21-23 and 42; and
detecting SARS-CoV-2 in the cultured cells.

Clause 20. The method of clause 18 or clause 19, wherein the amino acid sequence of the CXCR3 agonist peptide is at least 95% identical to any one of SEQ ID NOs: 7-9, 12-19, 21-23 and 42.

Clause 21. The method of any one of clauses 18-20, wherein the amino acid sequence of the CXCR3 agonist peptide comprises or consists of any one of SEQ ID NOs: 7-9, 12-19, 21-23 and 42.

The following examples are provided to illustrate certain particular features and/or implementations. These examples should not be construed to limit the disclosure to the particular features or implementations described.

### EXAMPLES

### Example 1: Expression of IP-10 in Human Organs Following SARS-CoV-2 Infection

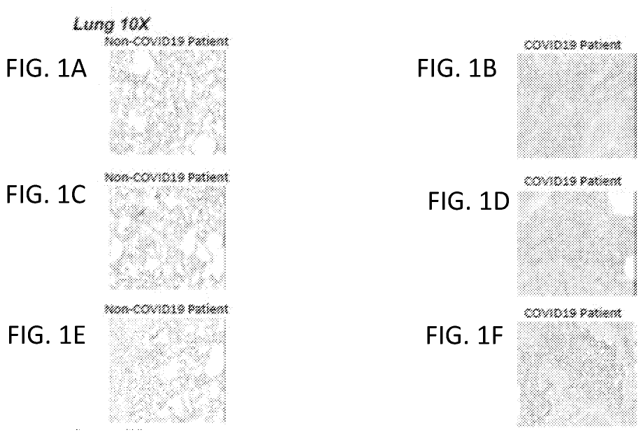

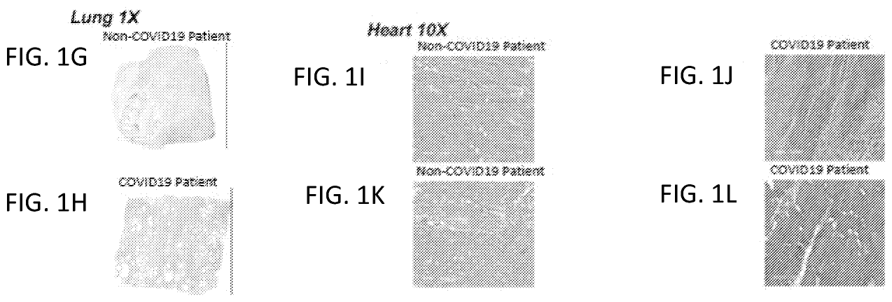

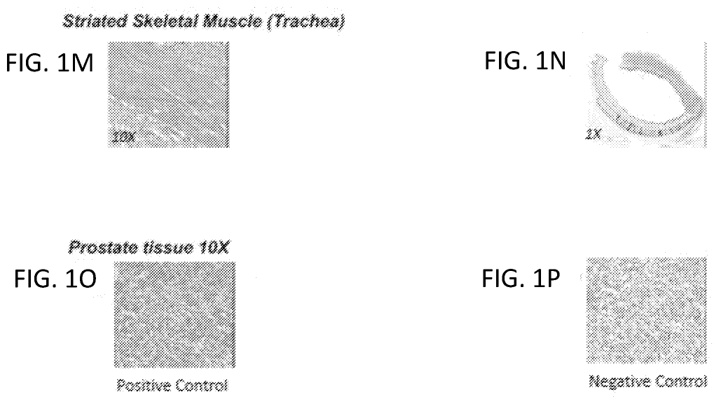

Expression of IP-10 in the lung, heart and skeletal muscle of a COVID-19 patient was compared to expression of IP-10 in the same organs of a non-COVID-19 subject. IP-10 protein was detected by immunohistochemistry (IHC) using a rabbit anti-IP-10 primary antibody and an anti-rabbit HRP-conjugated secondary antibody. The results demonstrated that IP-10 expression was increased in organs of the COVID-19 patient relative to the organs of the healthy subject (FIGS. 1A-1P).

### Example 2: Small Peptides that Act on Chemokine Receptor CXCR3 and/or Modulate CXCL10-CXCR3 Signaling

This example provides the sequences of 42 small peptides that function as CXCR3 agonists or antagonists. Four of the peptides are IP-10 peptides or variants thereof (SEQ ID NOs: 1-4). In addition, a series of small peptides (13 to 25 amino acids in length; SEQ ID NOs: 5-42) were developed by in silico prediction-based functional peptide design to directly bind to CXCL4, CXCL9, CXCL10, CXCL11, CXCR3, CXCR4 or DPP4. The amino acid sequences of each peptide are provided in Table 1.

The protein-protein interactions of the various receptors with the CXCR3 agonist/antagonist peptides were evaluated utilizing ClusPro (see Comeau et al., Nucl. Acids Res. 32: W96-99, W96-W99, DOI: 10.1093/nar/gkh354, 2004). This program is available on the internet at the Boston University website (nrc.bu.edu/cluster). The server performs three computational steps: (1) rigid body docking by sampling conformation; (2) root-mean-square deviation (RMSD) based on clustering of the 1,000 lowest energy structures generated to find the largest clusters that will represent the most likely models of the complex; and (3) refinement of the selected structures using energy minimization. This in silico method is predictive of the interactions between test peptides and selected receptors. The binding energy associated with the peptide's interaction with the site associated with dimerization of the receptor, and the binding energy associated with the peptide's interaction with the site associated with ligand binding to the receptor were determined to identify the peptides as agonists or antagonists (see US 2019/0298802, which is herein incorporated by reference in its entirety), as set forth in Table 1.

TABLE 1
Agonist and antagonist peptides

| | SEQ ID | | |
|---|---|---|---|
| Name | NO: | Peptide Sequence | |
| IP-10p | 1 | PESKAIKNLLKAVSKEMSKRSP | Agonist CXCL10 |
| IP-10-1 | 2 | PESKVIKNLLKVASKERSKRSP | Agonist CXCL10 |
| IP-10-2 | 3 | PESRVIKNLLRVVSKEKSRRSP | Agonist CXCL10 |
| IP-10-3 | 4 | FAKAIKNFAKAVAKFA | Agonist CXCL10 |
| CXCL10-1 | 5 | VRSTSISGGSISTSRV | Antagonist of CXCL10 |
| CXCL10-2 | 6 | IIPASQFGGFPQSAPII | Antagonist of CXCL10 |
| CXCL10-3 | 7 | FSPRVEIGGIEVRPSF | Both Antagonist and |
| | | | Agonist of CXCL10 |
| CXCL11-1 | 8 | FPMFKGGKFMPF | Antagonist of CXCL11 |
| CXCL11-2 | 9 | RLSLSIGGISLSR | Both Antagonist and |
| | | | Agonist of CXC11 |
| CXCR3LP2-1 | 10 | DDHKEINAGGANIEKHD | Antagonist of CXCR3 |
| CXCR3LP2-2 | 11 | DDHKEIGGIEKHD | Antagonist of CXCR3 |
| CXCL10-3-1 | 12 | FSPHVEIGGIEVHPSF | Antagonist of CXCL10 |
| CXCL10-3-2 | 13 | FTPHVEIGGIEVHPTF | Antagonist of CXCL10 |
| CXCL10-3-3 | 14 | FTHVEIGGIEVHTF | Both Antagonist and |
| | | | Agonist of CXCL10 |
| CXCL10-1MI | 15 | SISTSRVGGVRSTSIS | Antagonist of CXCL10 |
| CXCL10-2MI | 16 | FPQSAPIGGIIPASQF | Antagonist of CXCL10 |
| CXCL10-3MI | 17 | IEVRPSFGGFSPRVEI | Both Antagonist and |
| | | | Agonist of CXCL10 |
| CXCL11-1MI | 18 | KFMPFGGFPMFK | Antagonist of CXCL11 |
| CXCL11-2MI | 19 | ISLSRGGRLSLSI | Both Antagonist and |
| | | | Agonist of CXCL11 |
| CXCR3LP2-1MI | 20 | ANIEKHDGGDDHKEINA | Antagonist of CXCR3 |
| CXCL10-3-1MI | 21 | IEVHPSFGGFSPHVEI | Antagonist of CXCL10 |
| CXCL10-3-2MI | 22 | IEVHPTFGGFTPHVEI | Antagonist of CXCL10 |
| CXCL10-3-3MI | 23 | IEVHTFGGFTHVEI | Antagonist of CXCL10 |
| CXCR3LP2-2MI | 24 | EKHDIGGDDHKEI | Antagonist of CXCR3 |
| CR3-1 | 25 | DRFNSFRQPGGQRFSNFRD | Antagonist of CXCR4 |
| CR3-2 | 26 | FVWQVAARVGGVRAAVQWVF | Agonist of CXCR4 |
| CR3-3 | 27 | DGVQPFNYQGGQYNFPQVGD | Both Antagonist and |
| | | | Agonist of CXCR4 |
| CRF-4 | 28 | NDALAGLRMLIRGGRIMRLGALADN | Antagonist of CXCL10 |
| CRF-5 | 29 | LRMLIRGGRILMRL | Antagonist of CXCL10 |
| CR3-1-1 | 30 | DRFNSYRQPGGQRYSNFRD | Antagonist of CXCR4 |
| CR3-1-2 | 31 | DRYNSYRQPGGQRYSNYRD | Antagonist of CXCR4 |
| CR3-2-1 | 32 | FVWQVFARVGGVRAFVQWVF | Agonist of CXCR4 |
| CR3-2-2 | 33 | FVWFARYGGYRAFWVF | Antagonist of CXCR4 |
| CR3-3-1 | 34 | DGFQPFNYQGGQYNFPQFGD | Agonist of CXCR4 |
| CRF-4-1 | 35 | NDALFGLRMLIRGGRIMRLGFLADN | Agonist of CXCL10 |
| CRF-4-2 | 36 | NDWLFRMLIRGGRIMRFLWDN | Agonist of CXCL10 |
| CRF-4-3 | 37 | WLFRMWIRGGRWMRFLW | Agonist of CXCL10 |
| CRF-5-1 | 38 | LRFLIRGGRILFRL | Agonist of CXCL10 |
| CRF-5-2 | 39 | LRFLFRGGRFLFRL | Agonist of CXCL10 |
| CRF-5-3 | 40 | FRFLFRGGRFLFRF | Agonist of CXCL10 |
| CRF-5-4 | 41 | FRWLFRGGRFLWRF | Agonist of CXCL10 |
| CRF-5-5 | 42 | FRWLWRGGRWLWRF | Agonist of CXCL10 |

### Example 3: Correlation of SARS-CoV-2 Viral Load and CXCL10 Levels

This example describes studies to validate the correlation between viral load and CXCL10 levels in vitro, test CXCR3 agonist/antagonist peptides in a SARS-CoV-2 mouse model following infection by patient-authentic virus, and assess lung fibrosis.

CXCL10 is associated with a greater viral load in SARS-CoV-2 infection, which suggests that direct targeting of CXCL10 may inhibit COVID-19 specific disease pathogenesis. The small peptides (referred to herein as “FibroKine™ peptides”) described in Example 2 possess the ability to bind and either agonize or antagonize CXCL10.

Commercially available kits are employed to customize in vitro assays to assess CXCL10 levels in cell culture monolayers. Following the in vitro results, a study is conducted in SARS-CoV-2 infected mice to determine maximum tolerated dose (MTD) of the candidate FibroKine™ peptides and their protective effect to reduce fibrosis in liver and heart. These candidate FibroKine™ peptides are further tested to characterize their inhibitory concentration 50 (IC50) values in a hepatocyte culture. Moreover, MTT assays are conducted to determine in vitro compatibility of FibroKine™ peptides in hepatocyte and cardiomyocyte cultures.

The results are expected to show a positive correlation between SARS-CoV-2 viral load and CXCL10 levels and to lead to identification of efficacious FibroKine™ peptide to reduce tissue fibrosis in COVID-19 patients.

### Example 4: Dose Optimization Using an In Vivo SARS-CoV-2 Model

This example describes studies to elucidate absorption and distribution of FibroKine™ peptides in mice and perform dose optimization.

CXCL10 is a biomarker of COVID-19 disease and of increased inflammation across multiple systems in COVID-19, and may be indicative of disease outcomes. The present disclosure proposes CXCL10 as an important modulator of the hyperinflammatory state and a target for mitigating organ damage in COVID-19.

Studies are performed to evaluate efficacy of the FibroKine™ peptides identified in Example 3 for preventing tissue fibrosis in liver and heart at various peptides doses. In vivo SARS-CoV-2 animal studies are performed to monitor and assess inflammation continuously by plasma analysis and by histological analyses by sacrificing cohorts at 7-, 14-, and 21-days post infection. Further, absorption and distribution studies are conducted with single or repeated doses. Mice from Example 3 are used to determine the number of animals required to attain sufficient statistical power.

It is expected that these studies will determine the dose or dose-range where the selected FibroKine™ peptides are most efficacious. These studies are also expected to provide information to estimate the therapeutic range for the peptides. Additionally, it is expected that FibroKine™ peptides will prevent tissue fibrosis well below MTD and that the peptides will be absorbed readily from nasal mucosa and distributed in target organs at efficacious levels.

In view of the many possible implementations to which the principles of the disclosure may be applied, it should be recognized that the illustrated implementations are only examples and should not be taken as limiting the scope of the disclosure. Rather, the scope of the disclosure is defined by the following claims.

1. A method of treating a disease, disorder or syndrome associated with a severe acute respiratory syndrome coronavirus 2 (SARS-CoV-2) infection in a subject, comprising administering to the subject a therapeutically effective amount of a composition comprising at least one C-X-C chemokine receptor 3 (CXCR3) antagonist peptide, wherein the peptide is 12 to 30 amino acids in length and comprises at least 12 consecutive amino acids of any one of SEQ ID NOs: 1-6, 10, 11, 20 and 24-41.

2. The method of claim 1, wherein:
the amino acid sequence of the CXCR3 antagonist peptide is at least 95% identical to any one of SEQ ID NOs: 1-6, 10, 11, 20 and 24-41; or
the amino acid sequence of the CXCR3 antagonist peptide comprises or consists of any one of SEQ ID NOs: 1-6, 10, 11, 20 and 24-41.

3. (canceled)

4. The method of claim 1, further comprising selecting a subject with a current or prior SARS-CoV-2 infection for treatment.

5. The method of claim 4, wherein the prior SARS-CoV-2 infection occurred within the last six months.

6. The method of claim 1, wherein the subject has been diagnosed with coronavirus disease 19 (COVID-19).

7. The method of claim 1, wherein the subject has increased expression of IP-10.

8. The method of claim 1, wherein the disease, disorder or syndrome associated with infection by SARS-CoV-2 comprises sepsis, cytokine storm syndrome, cardiomyopathy, heart failure, respiratory failure, liver failure, kidney failure, multi-organ failure, fibrosis, pathological tissue remodeling, gastrointestinal inflammation, hypercoagulation, thrombocytopenia, thrombosis, ischemia, ischemia/reperfusion injury, conjunctivitis, keratitis, delirium, neuropathy, or any combination thereof.

9. The method of claim 8, wherein fibrosis is fibrosis of the heart, lung, kidney or skin.

10. The method of claim 1, wherein the disease, disorder or syndrome associated with infection by SARS-CoV-2 comprises heart failure.

11. The method of claim 1, wherein the composition comprises at least two, at least three, at least four or at least five CXCR3 antagonist peptides.

12. The method of claim 1, wherein the composition further comprises a carrier protein.

13. The method of claim 12, wherein the carrier protein comprises heparin.

14. The method of claim 1, wherein the composition further comprises a pharmaceutically acceptable carrier.

15. The method of claim 1, further comprising administering to the subject a monoclonal antibody specific for SARS-CoV-2.

16. The method of claim 15, wherein the monoclonal antibody comprises bamlanivimab, casirivimab and/or imdevimab.

17. The method of claim 1, further comprising administering to the subject remdesivir, dexamethasone, and/or supplemental oxygen.

18. An in vitro method for enhancing replication of severe acute respiratory syndrome coronavirus 2 (SARS-CoV-2) in a cell, comprising contacting the cell with an effective amount of a C-X-C chemokine receptor 3 (CXCR3) agonist peptide, wherein the peptide is 12 to 30 amino acids in length and comprises at least 12 consecutive amino acids of any one of SEQ ID NOs: 7-9, 12-19, 21-23 and 42.

19. The method of claim 18, wherein:
the amino acid sequence of the CXCR3 agonist peptide is at least 95% identical to any one of SEQ ID NOs: 7-9, 12-19, 21-23 and 42; or
the amino acid sequence of the CXCR3 agonist peptide comprises or consists of any one of SEQ ID NOs: 7-9, 12-19, 21-23 and 42.

20. (canceled)

21. A method for detecting the presence of severe acute respiratory syndrome coronavirus 2 (SARS-CoV-2) in a biological sample, comprising:
contacting cultured cells susceptible to infection by SARS-CoV-2 with the biological sample under conditions sufficient to allow for infection of the cultured cells if SARS-CoV-2 is present in the biological sample;
contacting the cultured cells with a C-X-C chemokine receptor 3 (CXCR3) agonist peptide, wherein the peptide is 12 to 30 amino acids in length and comprises at least 12 consecutive amino acids of any one of SEQ ID NOs: 7-9, 12-19, 21-23 and 42; and
detecting SARS-CoV-2 in the cultured cells.

22. The method of claim 21, wherein:
the amino acid sequence of the CXCR3 agonist peptide is at least 95% identical to any one of SEQ ID NOs: 7-9, 12-19, 21-23 and 42; or
the amino acid sequence of the CXCR3 agonist peptide comprises or consists of any one of SEQ ID NOs: 7-9, 12-19, 21-23 and 42.

23. (canceled)


SEQUENCE LISTING (42 sequences)

SEQ ID NO: 1 - protein, 22 aa, Artificial Sequence
  Synthetic peptide
PESKAIKNLLKAVSKEMSKRSP

SEQ ID NO: 2 - protein, 22 aa, Artificial Sequence
  Synthetic peptide
PESKVIKNLLKVASKERSKRSP

SEQ ID NO: 3 - protein, 22 aa, Artificial Sequence
  Synthetic peptide
PESRVIKNLLRVVSKEKSRRSP

SEQ ID NO: 4 - protein, 16 aa, Artificial Sequence
  Synthetic peptide
FAKAIKNFAKAVAKFA

SEQ ID NO: 5 - protein, 16 aa, Artificial Sequence
  Synthetic peptide
VRSTSISGGSISTSRV

SEQ ID NO: 6 - protein, 17 aa, Artificial Sequence
  Synthetic peptide
IIPASQFGGFPQSAPII

SEQ ID NO: 7 - protein, 16 aa, Artificial Sequence
  Synthetic peptide
FSPRVEIGGIEVRPSF

SEQ ID NO: 8 - protein, 12 aa, Artificial Sequence
  Synthetic peptide
FPMFKGGKFMPF

SEQ ID NO: 9 - protein, 13 aa, Artificial Sequence
  Synthetic peptide
RLSLSIGGISLSR

SEQ ID NO: 10 - protein, 17 aa, Artificial Sequence
  Synthetic peptide
DDHKEINAGGANIEKHD

SEQ ID NO: 11 - protein, 13 aa, Artificial Sequence
  Synthetic peptide
DDHKEIGGIEKHD

SEQ ID NO: 12 - protein, 16 aa, Artificial Sequence
  Synthetic peptide
FSPHVEIGGIEVHPSF

SEQ ID NO: 13 - protein, 16 aa, Artificial Sequence
  Synthetic peptide
FTPHVEIGGIEVHPTF

SEQ ID NO: 14 - protein, 14 aa, Artificial Sequence
  Synthetic peptide
FTHVEIGGIEVHTF

SEQ ID NO: 15 - protein, 16 aa, Artificial Sequence
  Synthetic peptide
SISTSRVGGVRSTSIS

SEQ ID NO: 16 - protein, 16 aa, Artificial Sequence
  Synthetic peptide
FPQSAPIGGIIPASQF

SEQ ID NO: 17 - protein, 16 aa, Artificial Sequence
  Synthetic peptide
IEVRPSFGGFSPRVEI

SEQ ID NO: 18 - protein, 12 aa, Artificial Sequence
  Synthetic peptide
KFMPFGGFPMFK

SEQ ID NO: 19 - protein, 13 aa, Artificial Sequence
  Synthetic peptide
ISLSRGGRLSLSI

SEQ ID NO: 20 - protein, 17 aa, Artificial Sequence
  Synthetic peptide
ANIEKHDGGDDHKEINA

SEQ ID NO: 21 - protein, 16 aa, Artificial Sequence
  Synthetic peptide
IEVHPSFGGFSPHVEI

SEQ ID NO: 22 - protein, 16 aa, Artificial Sequence
  Synthetic peptide
IEVHPTFGGFTPHVEI

SEQ ID NO: 23 - protein, 14 aa, Artificial Sequence
  Synthetic peptide
IEVHTFGGFTHVEI

SEQ ID NO: 24 - protein, 13 aa, Artificial Sequence
  Synthetic peptide
EKHDIGGDDHKEI

SEQ ID NO: 25 - protein, 19 aa, Artificial Sequence
  Synthetic peptide
DRFNSFRQPGGQRFSNFRD

SEQ ID NO: 26 - protein, 20 aa, Artificial Sequence
  Synthetic peptide
FVWQVAARVGGVRAAVQWVF

SEQ ID NO: 27 - protein, 20 aa, Artificial Sequence
  Synthetic peptide
DGVQPFNYQGGQYNFPQVGD

SEQ ID NO: 28 - protein, 25 aa, Artificial Sequence
  Synthetic peptide
NDALAGLRMLIRGGRIMRLGALADN

SEQ ID NO: 29 - protein, 14 aa, Artificial Sequence
  Synthetic peptide
LRMLIRGGRILMRL

SEQ ID NO: 30 - protein, 19 aa, Artificial Sequence
  Synthetic peptide
DRFNSYRQPGGQRYSNFRD

SEQ ID NO: 31 - protein, 19 aa, Artificial Sequence
  Synthetic peptide
DRYNSYRQPGGQRYSNYRD

SEQ ID NO: 32 - protein, 20 aa, Artificial Sequence
  Synthetic peptide
FVWQVFARVGGVRAFVQWVF

SEQ ID NO: 33 - protein, 16 aa, Artificial Sequence
  Synthetic peptide
FVWFARYGGYRAFWVF

SEQ ID NO: 34 - protein, 20 aa, Artificial Sequence
  Synthetic peptide
DGFQPFNYQGGQYNFPQFGD

SEQ ID NO: 35 - protein, 25 aa, Artificial Sequence
  Synthetic peptide
NDALFGLRMLIRGGRIMRLGFLADN

SEQ ID NO: 36 - protein, 21 aa, Artificial Sequence
  Synthetic peptide
NDWLFRMLIRGGRIMRFLWDN

SEQ ID NO: 37 - protein, 17 aa, Artificial Sequence
  Synthetic peptide
WLFRMWIRGGRWMRFLW

SEQ ID NO: 38 - protein, 14 aa, Artificial Sequence
  Synthetic peptide
LRFLIRGGRILFRL

SEQ ID NO: 39 - protein, 14 aa, Artificial Sequence
  Synthetic peptide
LRFLFRGGRFLFRL

SEQ ID NO: 40 - protein, 14 aa, Artificial Sequence
  Synthetic peptide
FRFLFRGGRFLFRF

SEQ ID NO: 41 - protein, 14 aa, Artificial Sequence
  Synthetic peptide
FRWLFRGGRFLWRF

SEQ ID NO: 42 - protein, 14 aa, Artificial Sequence
  Synthetic peptide
FRWLWRGGRWLWRF